(19) United States
(12) Plant Patent Application Publication
(10) Pub. No.: US 2015/0135378 P1
(43) Pub. Date: May 14, 2015

(54) Hop plant named 'ADHA 527'

(50) ;

(71) Applicant: Jason Perrault, Toppenish, WA (US)

(72) Inventor: Jason Perrault, Toppenish, WA (US)

(21) Appl. No.: 14/121,495

(22) Filed: Sep. 12, 2014

Related U.S. Application Data
(60) Provisional application No. 61/960,243, filed on Sep. 13, 2013.

(51) Int. Cl. A01H 5/00

(52) U.S. Cl. PLT/236

(57) ABSTRACT

A new hop plant named ADHA 527 is disclosed. The cones of ADHA 527 mature in mid to late September in Moxee, Wash., and yield a crop of 2200 to 2300 pounds per acre. ADHA 527 is used for its semi-dwarf size and aromatic quality.

### CROSS-REFERENCE TO RELATED APPLICATIONS

This application claims priority of co-pending provisional application Ser. No. 61/960,243.

### STATEMENT REGARDING FEDERALLY SPONSORED RESEARCH OR DEVELOPMENT

None

### GENUS AND SPECIES

Humulus lupulus

### VARIETY DENOMINATION

ADHA 527

### BACKGROUND OF THE INVENTION

‘ADHA 527’ is a product of a controlled breeding program carried out by the inventor in the Yakima Valley of Washington State. ADHA 527 was one of several seedlings resulting from cross number 05-072 made in 2005 with female parent Columbus (U.S. Plant Pat. No. 10,956) and male parent ADHA J3 (not patented). A single plant of ADHA 527 was selected in 2006. In 2009 that single plant was asexually propagated into 100 plants, which were planted in the area of Toppenish, Wash. These 100 plants were observed and evaluated for several years, and in 2012 were asexually propagated into a large scale test plot in Moxee, Wash. for further observation and evaluation. Throughout several generations of asexual propagation, ADHA 527 has been observed to retain its distinctive characteristics and has remained true to type.

### BRIEF DESCRIPTION OF THE PHOTOGRAPHS

FIG. 1 illustrates a mature ADHA 527 hop plant grown on a trellis;

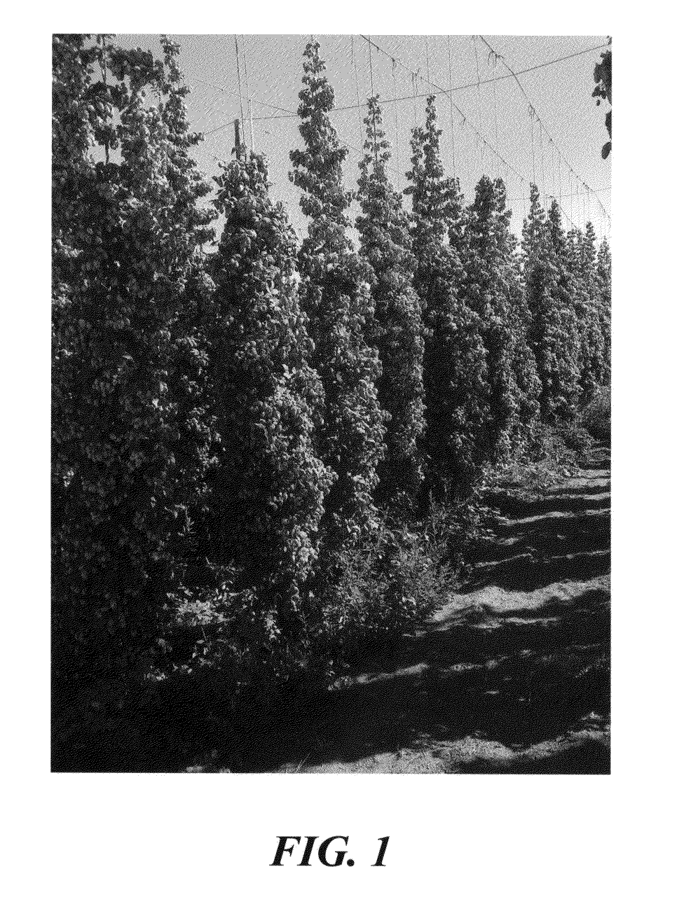

FIG. 2 illustrates whole cones and cross sections of cones of the ADHA 527 hop plant;

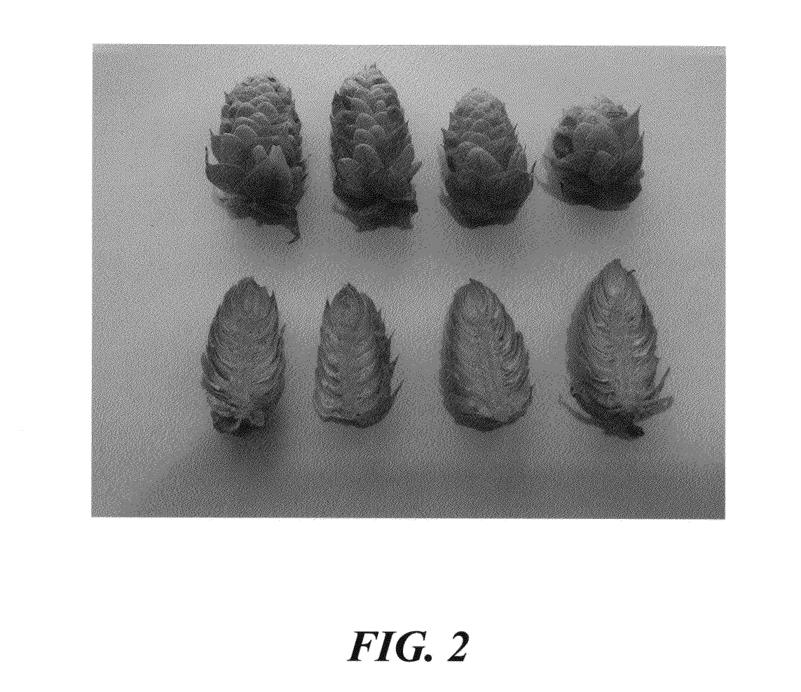

FIG. 3 illustrates the bine of a mature ADHA 527 hop plant; and.

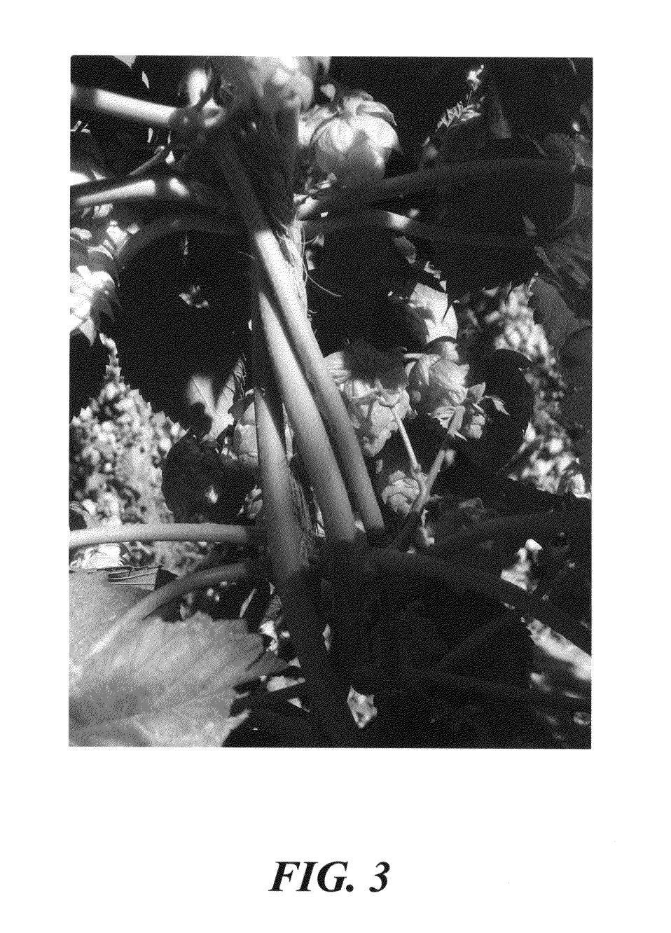

FIG. 4 illustrates the cones and leaves of a mature ADHA 527 hop plant.

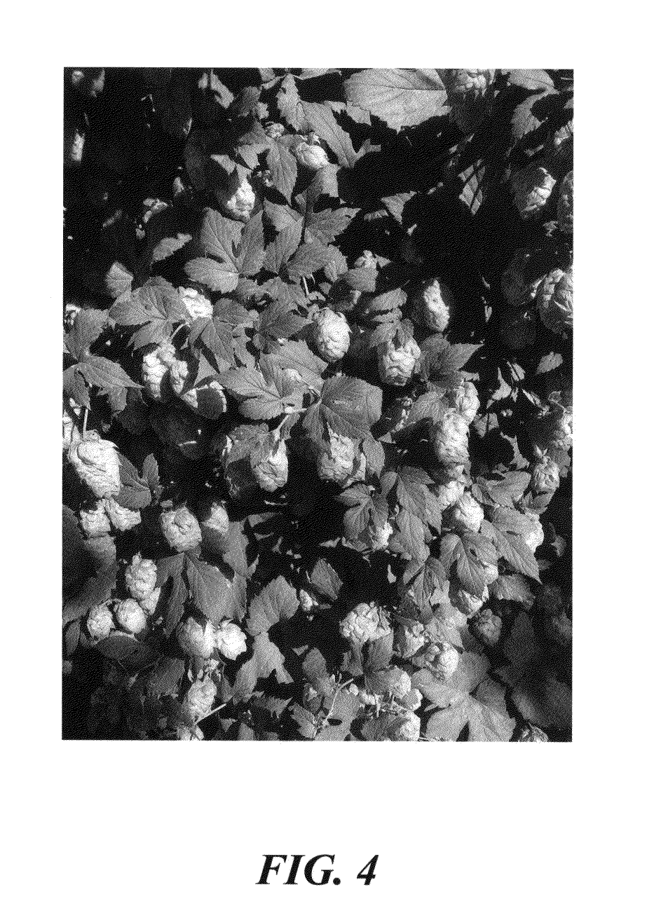

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2013 growing season at Moxee, Wash. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average. The color chart used was The Royal Horticultural Society Colour Chart, Copyright 2001, and all color observations were made outdoors in full sunlight.
Ploidy: Diploid
Use: Brewing beer.
Harvest date: September 15 to 25 during the 2009-2012 growing seasons, Moxee, Wash.
Crop yield: 2200 to 2300 pounds per acre
Disease susceptibility: Tolerant of both downy mildew and powdery mildew.
Pest susceptibility: Average susceptibility to pests, including hop aphids and two spotted spider mites.
Bine:
   Color.—Yellow Green 146C.
   Stripe.—Present, Yellow Green 152A.
   Bine diameter.—6.7 mm at base; 7.5 mm at 6 feet; and no measurement taken at terminal end.
Leaf:
   Color of mature leaf upper surface.—Green 147A.
   Color of mature leaf lower surface.—Green 147B.
   Number of lobes.—1-7.
   Margin.—Serrate.
   Serrations per inch.—4-5.
   Petiole color at base.—Yellow Green 144A.
   Vein color.—Green 147C.

1. What is claimed is a new and distinct hop plant as shown and described herein.
